(19) United States
(12) Patent Application Publication
(10) Pub. No.: US 2025/0269302 A1
(43) Pub. Date: Aug. 28, 2025

(54) TEST FILTER DEVICE

(71) Applicant: Zelva Capital LLC, San Antonio, TX (US)

(72) Inventors: Blake Branson, Austin, TX (US); Nathan Wicker, San Antonio, TX (US)

(21) Appl. No.: 19/066,407

(22) Filed: Feb. 28, 2025

Related U.S. Application Data
(60) Provisional application No. 63/559,050, filed on Feb. 28, 2024.

(51) Int. Cl. B01D 17/04; G01N 1/40; G01N 33/22

(52) U.S. Cl. CPC B01D 17/045; G01N 1/40; G01N 33/22

(57) ABSTRACT

Disclosed herein are example filtration test assemblies comprising an inner housing and an outer housing. The inner housing and outer housing define a fluid chamber between them, and a filtration element can be positioned in the fluid chamber. The devices disclosed herein can, in one example, separate or partially separate water from emulsions by passing the emulsions across the filtration element.

### CROSS-REFERENCE TO RELATED APPLICATION

This application claims benefit of U.S. Provisional Patent Application No. 63/599,050, filed on Feb. 28, 2024, which is hereby incorporated by reference.

### FIELD

The present disclosure relates generally to the field of filtration devices, and particularly for filtration devices for separating or partially separating a water-fuel emulsion to enable the testing of aviation fuel for impurities.

### BACKGROUND

The present disclosure relates generally to filtration devices for use in quality testing of fuels, particularly aviation grade kerosine fuels, diesel fuels, and military fuels. Such fuels may be sensitive to content of impurities such as surfactants left over from various manufacturing processes, and thus must be tested to ensure quality. More particularly, this disclosure pertains to various aspects of filtration devices used to characterize the levels of impurities in fuel samples, which rely on measuring the ability of a filter to extract water from an emulsion of fuel and water.

What is needed, then, are reliable, accurate, and repeatable devices for use in filtration testing of fuels for impurity content.

### BRIEF SUMMARY

Disclosed herein are example test filter devices for repeatably and with low variation measuring the presence of unwanted chemical inclusions in hydrocarbon fuels by emulsification and filtration. The test filter devices disclosed herein allow for improved control of geometric variables that affect test accuracy. According to some aspects of the present disclosure, the test filter devices can comprise an inner housing and an outer housing coupled together to define a fluid cavity, fluidly accessible through an entry channel and an exit channel. According to some aspects of the present disclosure, a filtration element can be positioned within the cavity between the entry channel and the exit channel. According to some aspects of the present disclosure, a shim can be included to fix or substantially fix the thickness of the fluid cavity and therefore of the filtration element positioned within the fluid cavity.

Certain examples include a test filter assembly. The test filter assembly comprises an external housing having an inlet end portion and an outlet end portion and comprising a first bore extending along a longitudinal axis. In some examples, test filter assembly also comprises an internal housing having an inlet end portion and an outlet end portion and positioned radially within the external housing and comprising a second bore extending along the longitudinal axis. In some examples, the test filter assembly also comprises a shim disposed between the external housing and the internal housing and a filter element disposed between the external housing and the internal housing. In some examples, the test filter assembly also includes a sealing element disposed between the external housing and the internal housing. The external housing the internal housing, and the sealing element define a fluid cavity disposed between the external housing and the internal housing. According to some examples, the fluid cavity contains the shim, and the filter element, and the first bore, the second bore, and the fluid cavity define a test throughput channel extending from an inlet end of the test filter assembly to an outlet end of the test filter assembly.

Numerous objects, features and advantages of the embodiments set forth herein will be readily apparent to those skilled in the art upon reading of the following disclosure when taken in conjunction with the accompanying drawings.

### BRIEF DESCRIPTION OF THE DRAWINGS

FIG. 1 is a top perspective view of a test filter assembly according to one aspect of the present disclosure.

FIG. 2 is a bottom perspective view of the test filter assembly of FIG. 1.

FIG. 3 is an exploded view of the test filter assembly of FIG. 1.

FIG. 4 is a longitudinal cross-sectional view of the test filter assembly of FIG. 1.

FIG. 5 is a bottom elevation view of the test filter assembly of FIG. 1.

FIG. 6 is a top elevation view of the test filter assembly of FIG. 1.

FIG. 7 is a lateral cross-sectional view of the test filter assembly of FIG. 1.

FIG. 8 is a chart showing the variation of filtration test results as a factor of filter gap.

### DETAILED DESCRIPTION

Reference will now be made in detail to aspects of the disclosure, one or more examples of which are illustrated in the drawings. Each example is provided by way of explanation, not limitation, of the disclosure. It will be apparent to those skilled in the art that various modifications and variations can be made to the examples presented in the present disclosure without departing from the scope or spirit thereof. For instance, features disclosed as part of one example can be used in conjunction with features of another example to yield a still further example. Thus, it is intended that the present disclosure covers such modifications and variations as come within the scope of the claims and their equivalents.

### General Terms

The word “exemplary” is used herein to mean “serving as an example, instance, or illustration.” Any implementation described herein as “exemplary” is not necessarily to be construed as preferred or advantageous over other implementations.

As used herein, the terms “first,” “second,” “third,” and so on may be used interchangeably to distinguish one component from another and are not intended to signify location or importance of the individual components.

The terms “coupled,” “fixed,” “attached to,” and the like refer both to direct coupling, fixing, or attaching, as well as indirect coupling, fixing, or attaching through one or more intermediate components or features, unless otherwise specified herein.

The singular forms “a,” “an,” and “the” include plural references, unless the context clearly indicates otherwise.

Unless otherwise indicated, all numbers expressing dimensions of components, percentages, temperatures, weights, and so forth, as used in the specification or claims are to be understood as being modified by the term “about.” Accordingly, unless otherwise indicated, implicitly or explicitly, the numerical parameters set forth are approximations that can depend on ordinary manufacturing tolerances, minor design variations, or limits of the measuring technique used. When directly and explicitly distinguishing embodiments from discussed prior art, the embodiment numbers are not approximates unless the word “about” is recited. Furthermore, not all alternatives recited herein are equivalents.

Approximating language, as used herein throughout the specification and claims, is applied to modify any quantitative representation that could permissibly vary without resulting in a change in the basic function to which it is related. Accordingly, a value modified by a term or terms, such as “about”, “approximately”, and “substantially”, are not to be limited to the precise value specified. In at least some instances, the approximating language may correspond to the precision of an instrument for measuring the value, or the precision of the methods or machines for constructing or manufacturing the components and/or systems. For example, the approximating language may refer to being within a 1, 2, 4, 10, 15, or 20 percent margin.

Here and throughout the specification and claims, range limitations are combined and interchanged, such ranges are identified and include all the sub-ranges contained therein unless context or language indicates otherwise. For example, all ranges disclosed herein are inclusive of the endpoints, and the endpoints are independently combinable with each other.

### Introduction to the Disclosed Technology

Disclosed herein are aspects of a test filter assembly for assessing the chemical composition of hydrocarbon fuel, including surfactant content in fuel samples. Generally, such test units comprise an outer housing and an inner housing coupled together to form a fluid cavity between the inner housing and outer housing. A filtration element is positioned in the cavity.

When a fluid is introduced to the fluid cavity, it will pass across the filter and partially separate. This can be used to test for chemical inclusions in the fluid, as chemical inclusions (for example, impurities left over from production of a chemical fluid) can degrade the performance of coalescing fuel filters. For example, such filtration units can be used to test for remnant surfactant content in hydrocarbon fuels by preparing an emulsion of water and the fuel to be tested, and measuring the separation characteristics of the emulsion, and particularly how effectively undissolved water can be removed from the emulsion by the filter element.

However, inventors have discovered that minor variations in the position and thickness of the filtration element, and/or the dimensions of the fluid cavity, can cause major deviations in the results of fluid separation, and therefore of measurements made using filtration units previously known in the art. Accordingly, there is a need for improved filtration units that remove or reduce variations in the cavity dimensions and/or the dimensions or placement of the filtration unit. Disclosed herein are various aspects of such devices that advantageously reduce such variations in dimensions and placement.

### Aspects of the Disclosed Technology

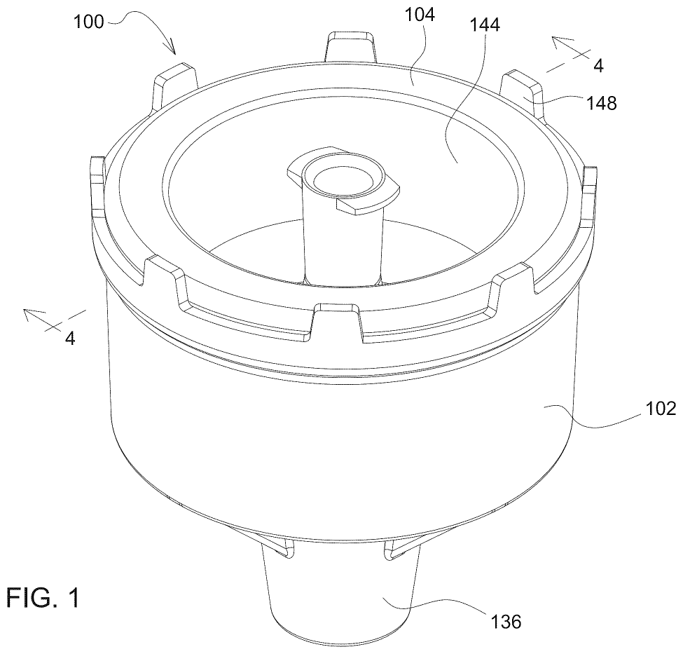

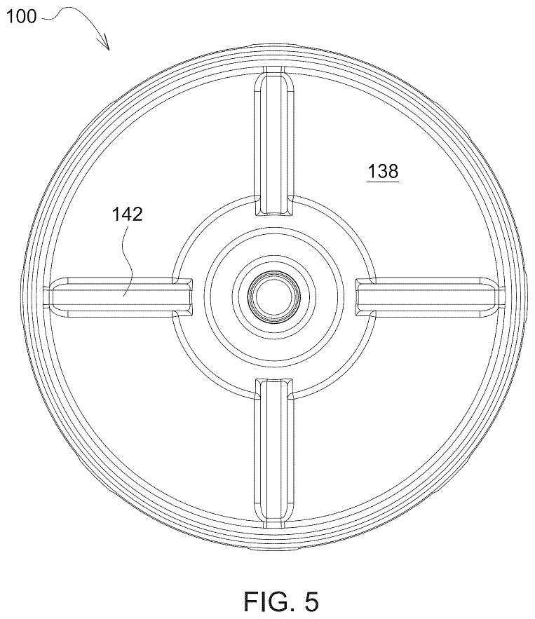

A test filter assembly 100 according to one aspect of the present disclosure is shown in FIGS. 1-7. The test filter assembly 100 includes an external housing 102 and an internal housing 104. The internal housing 104 can be nested inside the external housing 102 as shown in FIG. 1, to form the test filter assembly 100, such that, in the case of a test filter assembly 100 having a generally circular cross section as illustrated in FIG. 1, the external housing 102 and the internal housing 104 are concentric and coaxial.

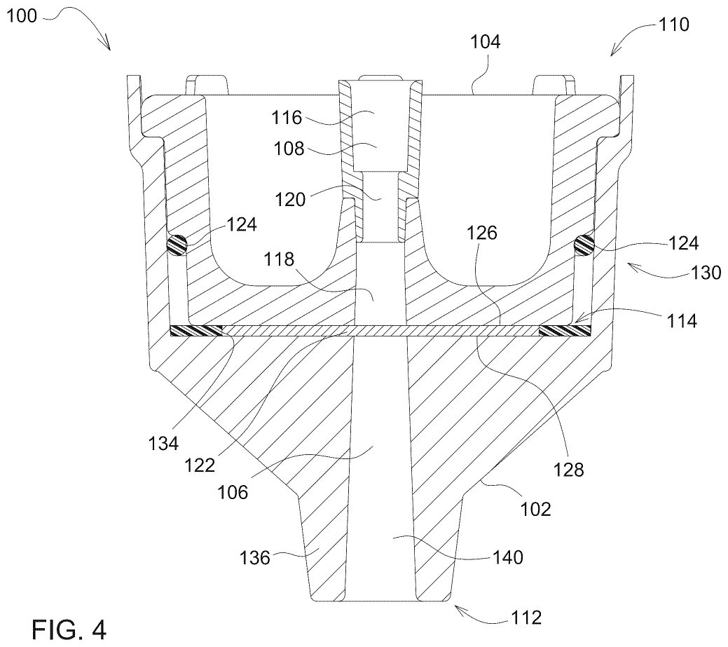

As illustrated in FIG. 4, the external housing 102 can comprise a first bore 106 extending therethrough along a longitudinal axis of the test filter assembly 100. Similarly, the internal housing 104 can comprise a second bore 108 extending therethrough along the longitudinal axis of the test filter assembly 100. As shown in FIG. 4, the first bore 106 and the second bore 108 can be axially aligned with one another so as to define a fluid pathway from an inlet end portion 110 of the test filter assembly 100 to an outlet end portion 112 of the test filter assembly 100.

With continued reference to FIG. 4, the external housing 102 and the internal housing 104 can also define or partially define a fluid chamber 114 positioned between the external housing 102 and the internal housing 104. The 114 is in fluid communication with the second bore 108 and the first bore 106, such that a fluid can be admitted to the test filter assembly 100 through the second bore 108 (that is, near the inlet end portion 110 of the test filter assembly 100), enter the fluid chamber 114 and exit the fluid chamber 114 through the first bore 106 (that is, from the outlet end portion 112 of the test filter assembly 100).

According to some aspects of the present disclosure, such as in the example illustrated in FIG. 4, the second bore 108 can further comprise an inlet end portion 116 an outlet end portion 118, and a central portion 120 extending between the inlet end portion 116 and the outlet end portion 118. According to some aspects of the present disclosure, the inlet end portion 116, the outlet end portion 118, and the central portion 120 can vary in diameter. For example, as shown in FIG. 4, the inlet end portion 116 can narrow in a taper as it extends away from the inlet end portion 110 of the test filter assembly 100 towards the central portion 120. The central portion 120 can have a smaller diameter than either the inlet end portion 116 or the outlet end portion 118 between which it extends, thereby providing a chokepoint for the flow of fluid from the inlet end portion 110 of the test filter assembly 100 as it passes through the second bore 108 towards the fluid chamber 114. As shown in FIG. 4, the outlet end portion 118 can also widen gradually in a taper as it extends from the central portion 120 towards the fluid chamber 114.

Taken together, the first bore 106, the second bore 108, and the fluid chamber 114 define a test throughput channel through which a fluid (such as a fuel-water emulsification) can flow through the test filter assembly 100.

Certain components such as seals or filtration elements can be contained within the fluid chamber 114. For example, as shown in FIG. 4, the test filter assembly 100 can further comprise a filtration element 122 and a sealing element 124. The filtration element 122 can be positioned between an external bottom surface 126 of the internal housing 104 and an internal bottom surface 128 of the external housing 102. As shown in FIG. 4, the filtration element 122 can be positioned so that fluid traversing the fluid chamber 114 from the outlet end portion 118 of the second bore 108 to the first bore 106 must pass across the filtration element 122, and thus may separate or partially separate into one or more constituent components. According to one aspect of the present disclosure, the filtration element 122 can be a coalescing filter, such as a fiberglass coalescing filter, however as will be appreciated by those skilled in the art, other suitable materials may be used for the separation or partial separation of fluids introduced into the fluid chamber 114 and passed across the filtration element 122.

According to one aspect of the present disclosure, the filtration element 122 may comprise a filter with more than one layer. For example, the filtration element 122 may comprise multiple layers of filter material, such as at least a first and second layer of filter material. In some examples, more than two layers of filter material may be present. The separate layers of filter material can be bonded or otherwise permanently attached to one another or left loose (that is, not bonded or permanently attached to one another) in various examples.

According to some aspects of the present disclosure, the sealing element 124 can also be positioned between the internal housing 104 and the external housing 102. For example, as shown in FIG. 4, the sealing element 124 is positioned towards an upper portion 130 of the fluid chamber 114. The sealing element 124 provides a fluid-tight seal between the external housing 102 and the internal housing 104 that prevents the undesired egress of fluids (such as, for example, a water and hydrocarbon fuel emulsion) from the fluid chamber 114. According to certain aspects of the present disclosure, the sealing element 124 can be an elastomeric ring or gasket, such as an O-ring, however any suitable material for forming a fluid tight seal between the external housing 102 and the 104 may be used in place of an elastomeric ring.

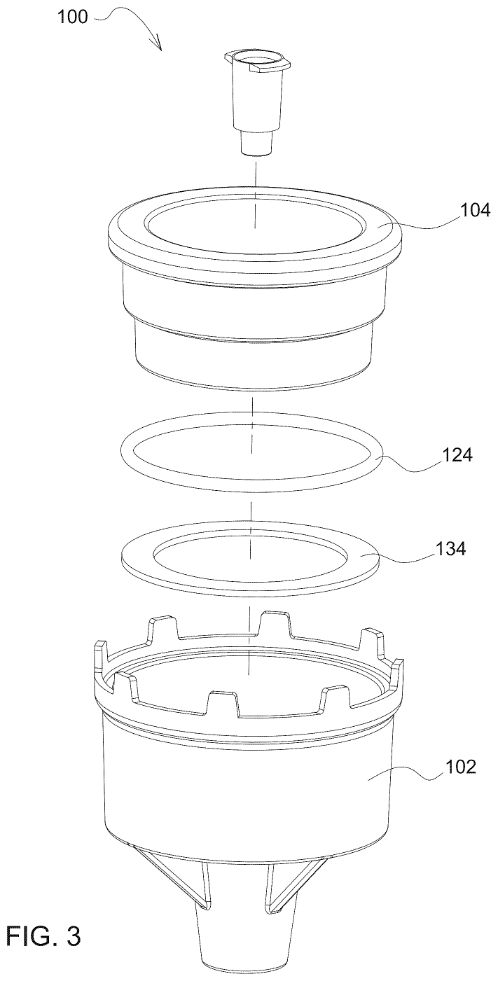

According to certain aspects of the present disclosure, the test filter assembly 100 can also comprise a shim 134, as shown in the exploded view of the test filter assembly 100 illustrated in FIG. 3. As shown in FIG. 4, the shim 134 can be positioned between the external housing 102 and the internal housing 104. In some examples, the shim 134 can be an annular ring which is disposed around the filtration element 122. In some examples, the shim 134 can extend from the outer diameter of the filtration element 122 to an inner diameter of the fluid chamber 114. Thereby, the shim 134 prevents lateral and/or radial slip of the filtration element 122 within the fluid chamber 114, and can ensure proper alignment between the filtration element 122 and both the first bore 106 and the second bore 108.

In some examples, the shim 134 may also ensure that the spacing between the external bottom surface 126 of the internal housing 104 and the internal bottom surface 128 of the external housing 102 is a desired distance, determined by the thickness of the shim 134. That is, in such examples, the shim 134 can limit a minimum axial thickness of the fluid chamber 114 in the axial direction by spacing the external housing 102 and the internal housing 104 axially apart from one another. Advantageously, this reduces variation in separation behavior when a fluid passes across the filtration element 122 which may arise from variation and/or tolerances in the sizing and relative positions of the external housing 102 and the internal housing 104, thereby controlling an axial spacing between the internal housing and the external housing. This in turn may improve the accuracy of any tests relying on the separation behavior of the fluid as it passes across the filtration element 122.

According to one aspect of the present disclosure, the shim 134 can have a thickness ranging from 0.02 inches to 0.03 inches, such as for example 0.021 inches, 0.022 inches, 0.023 inches, 0.024 inches, 0.025 inches, 0.026 inches, 0.027 inches, 0.028 inches, 0.029 inches, or 0.030 inches. It will be further appreciated, however that other thicknesses of shim may be used, such as shims thinner than 0.02 inches or shims thicker than 0.03 inches, depending on the desired thickness of the filtration element 122 and/or any desired compression of the filtration element 122 between the external housing 102 and the internal housing 104.

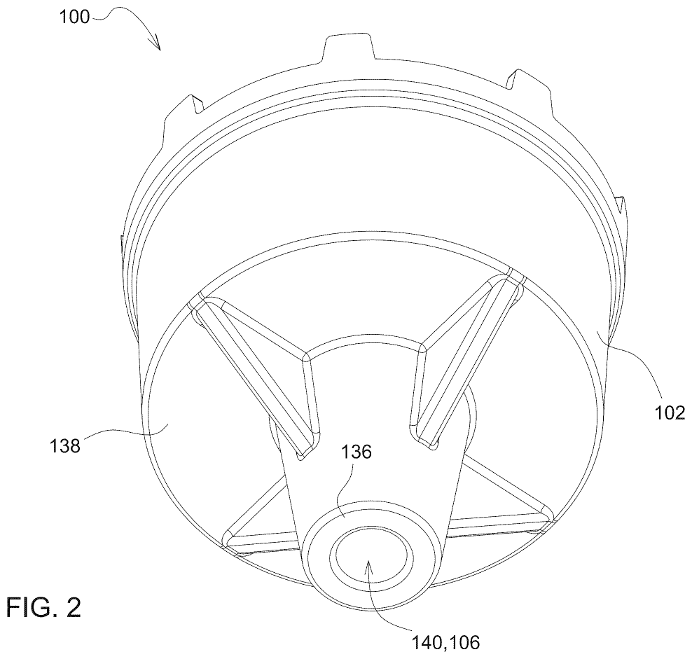

Turning now to FIG. 2, the test filter assembly 100 can also comprise a nozzle 136 which extends from an exterior bottom surface 138 of the external housing 102. The nozzle 136 can contain a downstream portion 140 of the first bore 106. The nozzle 136 can serve to direct an egress flow of the fluid from the fluid chamber 114 after it has passed across the filtration element 122. This can, for example, facilitate the collection of the filtrate exiting the test filter assembly 100 for subsequent measurements and/or sample collection.

According to some aspects of the present disclosure, the test filter assembly 100 may also comprise one or more fins 142 disposed along the exterior bottom surface 138 of the external housing 102. The one or more fins 142 can, in some examples, extend from the nozzle 136 towards an outer diameter or outer perimeter of the exterior bottom surface 138 of the external housing 102, as shown in FIG. 2. In such examples, the one or more fins 142 can serve to help align the test filter assembly 100 with a receiver device or container that is configured to receive the fluid after it passes through the test filter assembly 100.

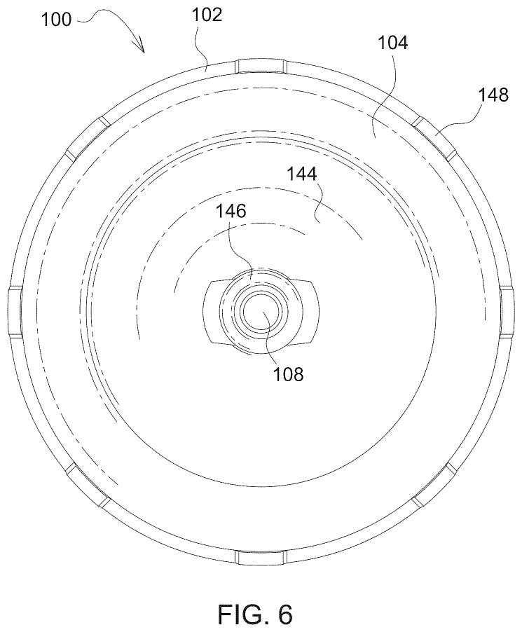

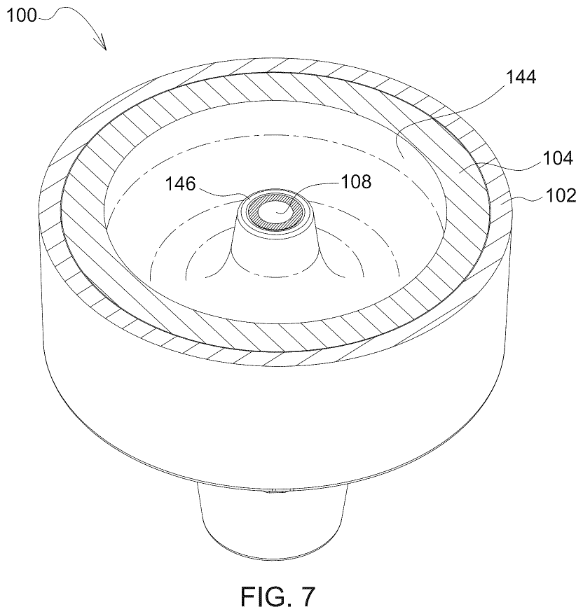

According to one aspect of the present disclosure, the internal housing 104 can also comprise an upper cavity portion 144, shown in FIGS. 6 and 7. The upper cavity portion 144 can have an annular geometry, and can fully surround the second bore 108 extending through the internal housing 104. In such examples, the upper cavity portion 144 defines an inlet nozzle 146. This configuration may be advantageous in examples where fluid, such as a fuel-water emulsion, is introduced to the test filter assembly 100 through a device that can fit over the inlet nozzle 146.

In some examples, the test filter assembly 100 can also comprise an adapter attached to the upstream end of the second bore 108 (that is, upstream of the inlet end portion 116). The adapter can serve to facilitate the connection of the test filter assembly 100 to an injector and/or a fluid source. In some examples, the adapter can comprise a threaded attachment point. The threaded attachment point can be internally threaded and configured to connect with an externally threaded connector portion of an injector or syringe, or can be externally threaded and configured to for threaded engagement with an internally threaded connector portion of an injector or a fluid delivery syringe.

According to one aspect of the present disclosure, the test filter assembly 100 can also comprise one or more crimping tabs 148 circumferentially disposed on the upstream end portion of the external housing 102, as shown in FIGS. 1 and 6. The one or more crimping tabs 148 can extend in the longitudinal direction from the upstream end portion of the external housing 102. One or more crimping tabs 148 may be deformed to secure the position of the inner housing 104 in relation to the external housing 102.

Also disclosed herein are methods for the use of test filter apparatus, such as the test filter assembly 100 disclosed herein and discussed in greater detail above and in reference to FIGS. 1-7. In one example, a test filter apparatus such as the test filter assembly 100 disclosed herein can be used to test for undesirable chemical content in fluid samples, such as in hydrocarbon fuels and particularly aviation fuels. In the case of such fuels, surfactant content left over from the production of the fuel may be undesirably retained in the fuel, and the content of such remnant surfactant chemicals can be measured, for example, by measuring the tendency of a fuel sample to retain emulsified water.

According to one general example, a fuel sample may be tested by first obtaining a fuel sample, such as a sample of a fuel having an unknown concentration of a surfactant, and blending the fuel sample with a quantity of water to produce an emulsion or suspension of fuel and water. Example useful methods for preparing such emulsions and/or suspensions of fuel and water are provided in ASTM standards D7224 and D3948, and will be readily understood by a person of ordinary skill in the art.

The fuel-water emulsion and/or suspension can then be partially filtered by passing it through a test filter apparatus, such as the test filter assembly 100 disclosed herein and discussed in greater detail above. For example, the fuel-water emulsion and/or suspension can be admitted to the test filter assembly 100 through the second bore 108, flow into the fluid chamber 114, pass across the filtration element 122, and exit the test filter assembly 100 through the first bore 106. As the fuel-water emulsion passes across the filtration element 122, a portion of the water entrained in the emulsion will coalesce and be released from the fuel component of the emulsion leaving a remainder portion of the water in the resulting filtered fuel-water emulsion.

Any presence of trace surfactant materials in the fuel sample will reduce the ability of the fuel sample to release entrained water when passing through a coalescing filter element, and therefore as surfactant content increases, an increasing amount of emulsified water will be retained in the fuel even after the sample has been passed across the filtration element 122. Because entrained water influences the optical properties of the fuel-water emulsion, (such as, for example, the optical transmissivity of the emulsion), and because the amount of water that remains entrained after filtration increases with increasing surfactant content, the surfactant content of the fuel can be calculated by measuring one or more optical properties of the emulsion, such as light transmissivity through the sample, after it has been filtered, and deriving a surfactant content from the measured optical properties, such as the measured light transmissivity value.

The results of such test methods, however, and specifically the fraction of the retained water released from the emulsion by passing it across the filtration element 122, have been discovered to rely also on the distance across the filtration element 122. Without being bound to any particular theory, it is presently believed that fluid passageways through a filter (such as, for example, a fiberglass filter) may be altered (for example, made wider or narrower) by compressing or decompressing the filter, which is typically bound on either side by the external housing 102 and the internal housing 104 of the test filter assembly 100, as previously discussed. Therefore, small variations in the components of a test filter apparatus such as the test filter assembly 100 can in turn cause geometric variations in the thickness of the fluid chamber 114 in which the filtration element 122 sits.

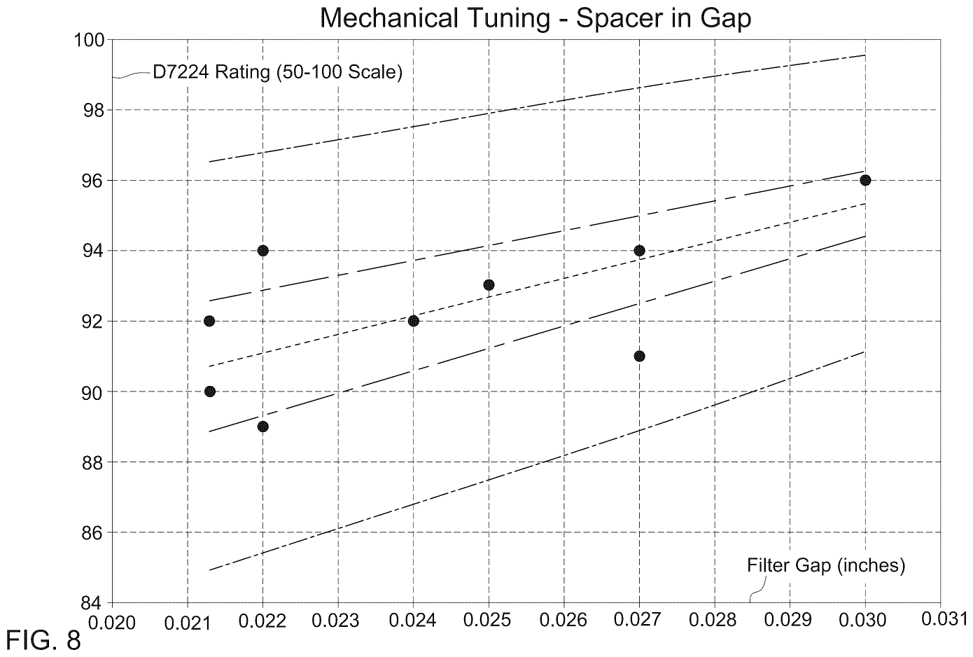

For example, for the test filter assembly 100, typical machining tolerances can routinely cause “slop” in the gap between the external housing 102 and the internal housing 104 in which the filtration element 122 sits on the order of 0.001 inches to 0.01 inches. The ASTM D7224 test, which converts light transmissivity results into a score on a scale ranging from 50-100, with values between 85 and 100 being a “passing” test for kerosene based aviation fuels. When such test filter apparatus is used in the ASTM D7224 test, the observed “slop” on the order of 0.001 inches to 0.01 inches can cause a 12-14 point scatter in the test, as demonstrated by using one or more shims of known dimension to alter gap distance to known values, while using identical filter and fuel samples, as presented in TABLE 1 and TABLE 2 below, and in FIG. 8.

TABLE 1
D7224 Rating as a Function of Gap Distance

| | FILTER HOUSING | D7224 |
|---|---|---|
| GAP SHIM STACK | GAP (in) | RATING |
| 0.010″ shim | 0.021 | 92 |
| 0.010″ + 0.005″ shim | 0.021 | 90 |
| 0.010″ + 0.010″ shim | 0.021 | 90 |
| 0.010″ + 0.002″ + 0.010″ shim | 0.022 | 94 |
| 0.010″ + 0.002″ + 0.010″ shim | 0.022 | 89 |
| 0.010″ + 0.002″ + 0.002″ + | 0.024 | 92 |
| 0.010″ shim | | |
| 0.010″ + 0.005″ + 0.010″ shim | 0.025 | 93 |
| 0.010″ + 0.005″ + 0.002″ + | 0.027 | 91 |
| 0.010″ shim | | |
| 0.010″ + 0.005″ + 0.002″ + | 0.027 | 94 |
| 0.010″ shim | | |
| 0.010″ + 0.010″ + 0.010″ shim | 0.030 | 96 |

TABLE 2
Statistical Analysis of D7224 Rating Test Results.

| Gap | | | | | | | | |
|---|---|---|---|---|---|---|---|---|
| Distance | D7224 | Curve fit | Approximater | | | Approximater | | |
| (in) | RATING | value | (reference fuel) | +½ r | −½ r | (field sample) | +½ r | −½ r |
| 0.021 | 90.7 | 90.7 | 3.7 | 92.6 | 89 | 11.6 | 96.5 | 84.9 |
| 0.022 | 91.5 | 91.1 | 3.4 | 92.8 | 89 | 11.3 | 96.8 | 85.4 |
| 0.024 | 92 | 92.1 | 3.2 | 93.7 | 91 | 10.7 | 97.5 | 86.8 |
| 0.025 | 93 | 92.7 | 2.8 | 94.1 | 91 | 10.4 | 97.9 | 87.5 |
| 0.027 | 92.5 | 93.7 | 3 | 95.2 | 92 | 9.8 | 98.6 | 88.9 |
| 0.03 | 96 | 95.3 | 1.6 | 96.1 | 95 | 8.4 | 99.5 | 91.1 |

Result variations arising merely from ordinary machining tolerances of the various parts of the test filter apparatus, such as are presently in use can, therefore, easily be enough to provide false positives or false negatives in test results. However, by fixing the gap with the inclusion of a spacer, such as the shim 134 of the test filter assembly 100 discussed herein, the variation in test results resulting from geometric variances in the gap distance (that is, the thickness of the fluid chamber 114) can be largely mitigated or even eliminated. Advantageously, this reduces the number of erroneous results generated while using a test filter apparatus such as the test filter assembly 100 disclosed herein.

### Additional Examples of the Disclosed Technology

This written description uses examples to illustrate the various aspects of the disclosed technology, and to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

Example 1. A test filter assembly comprising an external housing having an inlet end portion and an outlet end portion and comprising a first bore extending along a longitudinal axis; an internal housing having an inlet end portion and an outlet end portion and positioned radially within the external housing and comprising a second bore extending along the longitudinal axis; a shim disposed between the external housing and the internal housing; and a filter element disposed between the external housing and the internal housing; wherein the external housing and the internal housing define a fluid cavity disposed between the external housing and the internal housing, wherein the fluid cavity contains the shim, and the filter element, and wherein the first bore, the second bore, and the fluid cavity define a test throughput channel extending from an inlet end of the test filter assembly to an outlet end of the test filter assembly.

Example 2. The test filter assembly of any example herein, particularly example 1, wherein the first bore and the second bore are axially aligned with each other.

Example 3. The test filter assembly of any example herein, particularly example 1, further comprising a sealing element disposed between the external housing and the internal housing.

Example 4. The test filter assembly of any example herein, particularly example 3, wherein the sealing element is an elastomeric ring and is configured to prevent fluid from exiting or entering the fluid cavity except through the first bore or the second bore.

Example 5. The test filter assembly of any example herein, particularly example 1, wherein the external housing further comprises a centrally positioned nozzle extending longitudinally from an external bottom surface of the external housing, and wherein the first bore extends through the nozzle.

Example 6. The test filter assembly of any example herein, particularly example 5, further comprising one or more fins disposed on the external bottom surface of the external housing, the one or more fins radially extending from the nozzle towards an outer perimeter of the external housing.

Example 7. The test filter assembly of any example herein, particularly example 1, wherein the shim is an annular ring, and wherein the filter element is contained within the annular ring of the shim.

Example 8. The test filter assembly of any example herein, particularly example 1, wherein the filter element comprises at least a first filter layer and a second filter layer.

Example 9. The test filter assembly of any example herein, particularly example 8, wherein the first filter layer and the second filter layer are not permanently attached to one another.

Example 10. The test filter assembly of any example herein, particularly example 1, wherein the second bore comprises an upstream end portion, a downstream end portion, and a central portion between the upstream end portion and the downstream end portion, and wherein the central portion has a smaller diameter than the upstream end portion and the downstream end portion.

Example 11. The test filter assembly of any example herein, particularly example 1, wherein when the test filter assembly is in a fully assembled configuration, a bottom surface of the internal housing urges the shim into contact with an internal bottom surface of the external housing.

Example 12. The test filter assembly of any example herein, particularly example 1, further comprising a receiver portion positioned upstream of the second bore, the receiver portion comprising an external thread configured for threaded engagement with an internal thread of a fluid delivery syringe.

Example 13. The test filter assembly of any example herein, particularly example 1, wherein when the test filter is in a fully assembled configuration, a fluid passing between the second bore and the first bore must pass across the filter element.

Example 14. The test filter assembly of any example herein, particularly example 1 further comprising an adapter attached to an upstream end of the second bore.

Example 15. The test filter assembly of any example herein, particularly example 1, wherein the filter element is a fiberglass filter.

Example 16. The test filter assembly of any example herein, particularly example 1, wherein the shim limits a minimum axial thickness of the fluid cavity to a thickness ranging from 0.02 inches to 0.03 inches.

Example 17. A method for testing a fuel sample, using a filter assembly comprising an external housing with a first bore extending therethrough and an internal housing with a second bore extending therethrough, wherein the first bore and the second bore are in fluid communication with a filter element disposed therebetween; the method comprising obtaining fuel sample, wherein the fuel sample contains an unknown concentration of a surfactant; blending the fuel sample with a quantity of water to produce a fuel-water emulsion; admitting the fuel-water emulsion into the second bore; passing the fuel-water emulsion from the second bore to the first bore cross the filter element to release a portion of the quantity of water from the fuel-water emulsion while leaving a remainder portion of the quantity of water is retained in a resulting filtered fuel-water emulsion; measuring one or more optical properties of the filtered fuel-water emulsion; and calculating, from the one or more measured optical properties of the filtered fuel-water emulsion, the concentration of the surfactant in the fuel sample.

Example 18. The method of any example herein, particularly example 17, wherein the one or more optical properties includes optical transmissivity, and the optical transmissivity is used to calculate an amount of water in the remainder portion of the quantity of water, and the amount of water in the remainder portion of the quantity of water is further used to calculate the concentration of the surfactant in the fuel sample.

Example 19. The method of any example herein, particularly example 17, further comprising controlling an axial spacing between the internal housing and the external housing by introducing a shim to axially separate the internal housing from the external housing.

Example 20. The method of any example herein, particularly example 19, wherein the shim has a thickness ranging from 0.02 inches to 0.03 inches.

In view of the many possible aspects to which the principles of the disclosure can be applied, it should be recognized that the illustrated aspects are only preferred examples of the disclosure and should not be taken as limiting the scope of the disclosure. Rather, the scope of the disclosure is defined by the following claims. We therefore claim as our disclosure all that comes within the scope these claims.

What is claimed is:

1. A test filter assembly comprising:
an external housing having an inlet end portion and an outlet end portion and comprising a first bore extending along a longitudinal axis;
an internal housing having an inlet end portion and an outlet end portion and positioned radially within the external housing and comprising a second bore extending along the longitudinal axis;
a shim disposed between the external housing and the internal housing; and
a filter element disposed between the external housing and the internal housing;
wherein the external housing and the internal housing define a fluid cavity disposed between the external housing and the internal housing, wherein the fluid cavity contains the shim, and the filter element, and wherein the first bore, the second bore, and the fluid cavity define a test throughput channel extending from an inlet end of the test filter assembly to an outlet end of the test filter assembly.

2. The test filter assembly of claim 1, wherein the first bore and the second bore are axially aligned with each other.

3. The test filter assembly of claim 1, further comprising a sealing element disposed between the external housing and the internal housing.

4. The test filter assembly of claim 3, wherein the sealing element is an elastomeric ring and is configured to prevent fluid from exiting or entering the fluid cavity except through the first bore or the second bore.

5. The test filter assembly of claim 1, wherein the external housing further comprises a centrally positioned nozzle extending longitudinally from an external bottom surface of the external housing, and wherein the first bore extends through the nozzle.

6. The test filter assembly of claim 5, further comprising one or more fins disposed on the external bottom surface of the external housing, the one or more fins radially extending from the nozzle towards an outer perimeter of the external housing.

7. The test filter assembly of claim 1, wherein the shim is an annular ring, and wherein the filter element is contained within the annular ring of the shim.

8. The test filter assembly of claim 1, wherein the filter element comprises at least a first filter layer and a second filter layer.

9. The test filter assembly of claim 8, wherein the first filter layer and the second filter layer are not permanently attached to one another.

10. The test filter assembly of claim 1, wherein the second bore comprises an upstream end portion, a downstream end portion, and a central portion between the upstream end portion and the downstream end portion, and wherein the central portion has a smaller diameter than the upstream end portion and the downstream end portion.

11. The test filter assembly of claim 1, wherein when the test filter assembly is in a fully assembled configuration, a bottom surface of the internal housing urges the shim into contact with an internal bottom surface of the external housing.

12. The test filter assembly of claim 1, further comprising a receiver portion positioned upstream of the second bore, the receiver portion comprising an external thread configured for threaded engagement with an internal thread of a fluid delivery syringe.

13. The test filter assembly of claim 1, wherein when the test filter is in a fully assembled configuration, a fluid passing between the second bore and the first bore must pass across the filter element.

14. The test filter assembly of claim 1 further comprising an adapter attached to an upstream end of the second bore.

15. The test filter assembly of claim 1, wherein the filter element is a fiberglass filter.

16. The test filter assembly of claim 1, wherein the shim limits a minimum axial thickness of the fluid cavity to a thickness ranging from 0.02 inches to 0.03 inches.

17. A method for testing a fuel sample, using a filter assembly comprising an external housing with a first bore extending therethrough and an internal housing with a second bore extending therethrough, wherein the first bore and the second bore are in fluid communication with a filter element disposed therebetween; the method comprising:
obtaining fuel sample, wherein the fuel sample contains an unknown concentration of a surfactant;
blending the fuel sample with a quantity of water to produce a fuel-water emulsion;
admitting the fuel-water emulsion into the second bore;
passing the fuel-water emulsion from the second bore to the first bore cross the filter element to release a portion of the quantity of water from the fuel-water emulsion while leaving a remainder portion of the quantity of water is retained in a resulting filtered fuel-water emulsion;
measuring one or more optical properties of the filtered fuel-water emulsion; and
calculating, from the one or more measured optical properties of the filtered fuel-water emulsion, the concentration of the surfactant in the fuel sample.

18. The method of claim 17, wherein the one or more optical properties includes optical transmissivity, and the optical transmissivity is used to calculate an amount of water in the remainder portion of the quantity of water, and the amount of water in the remainder portion of the quantity of water is further used to calculate the concentration of the surfactant in the fuel sample.

19. The method of claim 17, further comprising controlling an axial spacing between the internal housing and the external housing by introducing a shim to axially separate the internal housing from the external housing.

20. The method of claim 19, wherein the shim has a thickness ranging from 0.02 inches to 0.03 inches.
